(19) United States
(12) Patent Application Publication
(10) Pub. No.: US 2025/0145951 A1
(43) Pub. Date: May 8, 2025

(54) COMPOSITIONS AND METHODS FOR EXPANDING LYMPHOCYTES

(71) Applicant: StemCell Technologies Canada Inc., Vancouver (CA)

(72) Inventors: Hitesh ARORA, Vancouver (CA); Mark BROWN, Vancouver (CA)

(21) Appl. No.: 18/837,739

(22) PCT Filed: Feb. 16, 2023
(86) PCT No.: PCT/CA2023/050200

§ 371 Date: Aug. 12, 2024

Related U.S. Application Data
(60) Provisional application No. 63/310,778, filed on Feb. 16, 2022.

(51) Int. Cl. C12N 5/0783; C12N 5/0781

(52) U.S. Cl. CPC C12N 5/0636; C12N 5/0635; C12N 5/0646; C12N 2500/36; C12N 2501/2302; C12N 2501/2304; C12N 2501/2306; C12N 2501/231; C12N 2501/2321; C12N 2501/52; C12N 2533/52; C12N 2533/54; C12N 2533/90

(57) ABSTRACT

Disclosed are media, supplements and methods for expanding mammalian lymphocytes or progenitors thereof, such as B cells, T cells, or NK cells. The disclosed media, supplements and methods may be used to expand mammalian lymphocytes or progenitors thereof plated as single cells, or at a clonal cell density. In some embodiments, the disclosed media, supplements and methods may also differentiate/activate the lymphocytes during expansion. The media, supplements and methods of this disclosure may be used in serum-free and feeder-free culture workflows.

### RELATED APPLICATION

This application claims the benefit of U.S. Provisional Patent Application No. 63/310,778, filed Feb. 16, 2022, the entire content of which is hereby incorporated by reference in its entirety.

### TECHNICAL FIELD

This disclosure relates to cell culture applications, and more specifically to cell culture applications using hematopoietic cells, and still more specifically to cell culture applications with population(s) of lymphocytes.

### BACKGROUND

The blood of mammals is composed of various cell types, including lymphocytes, thrombocytes, erythrocytes, and the direct and indirect precursors thereof. Lymphocytes may be referred to as white blood cells, and these function in the immune system of the host. Lymphocytes can be further subdivided into B cells, T cells, NK cells, macrophages, eosinophils, basophils, and neutrophils.

Lymphocytes, as with other blood cells, derive from a hematopoietic stem cell (HSC) that is capable of self-renewal and differentiating to each blood cell lineage. T cells are central components of adaptive immunity, NK cells serve important roles in innate immunity, and B cells are central components of humoral immunity to secrete antibodies upon binding an antigen.

Given the involvement of lymphocytes in sensing antigens in their environment and, upon stimulation, to illicit an immune response, they are subject of intense research and therapeutic interest. Specific B, NK, or T cell clones may be of particular interest, because of their ability to detect specific antigens or elicit a response, but it is exceedingly difficult to isolate such cells from the background of cells present in a blood sample. Engineering B, NK, or T cells to perform specific functions is a relatively newer approach to solve the isolation problem.

Even if the challenges of obtaining an individual, specific lymphocyte clone are overcome, a separate problem of expanding such cell to meaningful numbers emerges. Since lymphocytes plated as single cells or at a clonal density do not readily expand to significant enough numbers in vitro or ex vivo, there is a need for reagents and processes to clonally expand single lymphocytes (e.g. B, NK or T cells).

### SUMMARY

The present disclosure relates to supplements, culture medium, and methods for expanding mammalian lymphocytes, such as B, NK, or T cells, or progenitors thereof.

In one aspect of this disclosure are provided cell culture media supplements for expanding mammalian lymphocytes (such as B, NK, or T cells) or progenitors thereof. The supplements of this disclosure may comprise one or more of a ligand of CD40, a mixture of lipids, one or more cytokines, and a diluent. In one embodiment, a supplement of this disclosure may comprise two or more of the ligand of CD40, the mixture of lipids, and one or more cytokines.

In one embodiment, the diluent is aqueous. In one embodiment, the diluent is a buffer.

In one embodiment, a supplement of this disclosure may further comprise a first type of an extracellular matrix protein. In one embodiment, a supplement of this disclosure may further comprise a second type of an extracellular matrix protein. In one embodiment, a supplement of this disclosure may further comprise a third type of an extracellular matrix protein. In one embodiment, any one or all of the extracellular matrix protein(s) is/are recombinant. In one embodiment, any one or all of the extracellular matrix protein(s) is/are solubilized in a diluent.

In one embodiment, the extracellular matrix protein is selected from the group consisting of a collagen, an ECM1, an E-Cadherin, a laminin, an osteopontin, a fibronectin, a vitronectin, or a SPARC. In embodiments comprising more than one type of extracellular matrix protein, each type is different.

In one embodiment, a concentration of the first type of an extracellular matrix protein, the second type of an extracellular matrix protein (if present), or the third type of an extracellular matrix protein (if present) ranges between about 1 μg/ml to 1 mg/mL.

In one embodiment, the ligand of CD40 is a ligand of human or mouse CD40. In one embodiment, the ligand of CD40 comprises one or more proteins.

In one embodiment, the one or more cytokines are selected from IL-2, IL-4, IL-6, IL-10, and IL-21. In one embodiment, each of the foregoing cytokines are comprised in the supplement. In one embodiment, IL-2 and IL-10, and optionally IL-21, are comprised in the supplement.

In one embodiment, the mixture of lipids is chemically defined. In one embodiment, the mixture of lipids is animal component free. In one embodiment, the mixture of lipids stabilized. In one embodiment, the supplement is combined with a basal medium.

In one embodiment, the supplement is animal component-free and/or serum-free.

In one embodiment, the lymphocytes (e.g. T, NK or B cells) or progenitors thereof are seeded as single cells or at a clonal density.

In one embodiment, the supplement is concentrated. In one embodiment, the supplement is a 10× or greater concentration.

In another aspect of this disclosure are provided media for expanding mammalian lymphocytes (e.g. T, NK, or B cells) or progenitors thereof. Media of this disclosure may comprise a basal medium and one or more of: a ligand of CD40, a mixture of lipids, and one or more cytokines. Thus, in another aspect basal media and one or more supplements may be comprised in a kit. In one embodiment, the medium does not come into contact with a surface-bound or immobilized extracellular matrix protein.

In one embodiment, media of this disclosure may further comprise two or more of the ligand of CD40, the mixture of lipids, and the one or more cytokines.

In one embodiment, media of this disclosure may further comprise a first type of an extracellular matrix protein. In one embodiment, media of this disclosure may further comprise a second type of an extracellular matrix protein. In one embodiment, media of this disclosure may further comprise a third type of an extracellular matrix protein. In one embodiment, the extracellular matrix protein(s) is/are recombinant.

In one embodiment, the extracellular matrix protein is selected from the group consisting of a collagen, an ECM1, an E-Cadherin, a laminin, an osteopontin, a fibronectin, a vitronectin, or a SPARC. In embodiments comprising more than one type of extracellular matrix protein, each type is different.

In one embodiment, a concentration of the first type of an extracellular matrix protein, the second type of an extracellular matrix protein (if present), or the third type of an extracellular matrix protein (if present) ranges between about 1 μg/ml to 1 mg/mL.

In one embodiment, media of this disclosure may further comprise one or more cytokines. In one embodiment, the one or more cytokines are selected from IL-2, IL-4, IL-6, IL-10, and IL-21. In one embodiment, each of the foregoing cytokines are comprised in the supplement. In one embodiment, IL-2 and IL-10, and optionally IL-21, are comprised in the supplement.

In one embodiment, the ligand of CD40 is a ligand of human or mouse CD40. In one embodiment, the ligand of CD40 comprises one or more proteins.

In one embodiment, the mixture of lipids is chemically defined. In one embodiment, the mixture of lipids is animal component free. In one embodiment, the mixture of lipids stabilized.

In one embodiment, the lymphocytes (e.g. T, NK or B cells) or progenitors thereof are seeded as single cells or at a clonal density.

In one embodiment, the medium supports feeder-free expansion of lymphocytes and progenitors thereof.

In one embodiment, the medium supports animal component-free expansion of lymphocytes and progenitors thereof. In one embodiment, the culture medium is serum-free.

In another aspect of this disclosure are provided methods of expanding mammalian lymphocyte (e.g. T, NK, or B cells) or progenitors thereof. In one embodiment, the mammalian lymphocytes or progenitors thereof may be plated as single cells or at a clonal density. In one embodiment, the mammalian lymphocytes or progenitors thereof are not plated as single cells or at a clonal density.

The methods may comprise seeding lymphocytes or progenitors thereof into a culture environment comprising one or more of a ligand of CD40, a mixture of lipids, and one or more cytokines, and incubating/culturing the seeded cells or progenitors thereof in the culture environment for more than one day. In one embodiment, incubating/culturing the cells may comprise expanding the cells. In one embodiment, the cells may also differentiate and/or activate during the culturing/incubating step.

In one embodiment, the methods of this disclosure may further comprise a first type of an extracellular matrix protein in the culture environment. In one embodiment the methods of this disclosure may further comprise a second type of an extracellular matrix protein in the culture environment. In one embodiment the methods of this disclosure may further comprise a third type of an extracellular matrix protein in the culture environment.

In one embodiment, the first type of an extracellular matrix protein and/or the second type of an extracellular matrix protein (if present) and/or the third type of an extracellular matrix protein (if present) are comprised in a culture medium.

In one embodiment, the extracellular matrix protein is selected from the group consisting of a collagen, an ECM1, an E-Cadherin, a laminin, an osteopontin, a fibronectin, a vitronectin, or a SPARC. In embodiments comprising more than one type of extracellular matrix protein, each type is different.

In one embodiment, a concentration of the first type of an extracellular matrix protein, the second type of an extracellular matrix protein (if present), or the third type of an extracellular matrix protein (if present) ranges between about 1 μg/mL to 1 mg/mL.

In one embodiment, media of this disclosure may further comprise one or more cytokines. In one embodiment, the one or more cytokines are selected from IL-2, IL-4, IL-6, IL-10, and IL-21. In one embodiment, each of the foregoing cytokines are comprised in the supplement. In one embodiment, IL-2 and IL-10, and optionally IL-21, are comprised in the supplement.

In one embodiment, the ligand of CD40 is a ligand of human or mouse CD40. In one embodiment, the ligand of CD40 comprises one or more proteins.

In one embodiment, the mixture of lipids is chemically defined. In one embodiment, the mixture of lipids is animal component free. In one embodiment, the mixture of lipids stabilized.

In one embodiment, the seeding and incubating steps are in feeder-free conditions. In one embodiment, the seeding and incubating steps are in serum-free conditions. In one embodiment, the seeding and incubating steps are in animal component-free conditions. In one embodiment, the seeding and incubating steps are in each of the foregoing conditions.

In one embodiment, the lymphocytes or progenitors thereof are human or mouse.

In one embodiment, the lymphocytes are human or mouse B cells, and the B cells are memory B cells or plasmablasts.

In one embodiment, the methods of this disclosure may further comprise differentiating the lymphocytes or progenitors thereof in the culture environment. In one embodiment, the methods of this disclosure may further comprise activating and/or differentiating the lymphocytes or progenitors thereof in the culture environment.

In one embodiment, the incubating step is for at least 3 days.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 shows a bar graph of marker expression among cells of leukapheresis samples either before (light grey) or after (dark grey) enrichment of B lineage cells.

FIG. 2 shows representative images of B lineage cell expansion over time when plated as single cells. Single B cells from a donor were seeded in a control condition that did not include exogenously-added extracellular matrix protein diluted in culture medium and in various media conditions that included different combinations of exogenously-added extracellular matrix proteins. Conditions 1, 2, and 3 included different pairwise combinations of three extracellular matrix proteins. Condition 4 included all three extracellular matrix proteins. Single cells on day 0 are circled.

FIG. 3 shows representative images of B lineage cell expansion when plated as single cells. Single B cells from a different donor than in FIG. 2 were expanded in conditions that included two of the exogenously-added extracellular matrix proteins from FIG. 2 diluted individually in culture medium (Conditions 1 and 2) and in combination (Condition 3). Scale bars represent 200 μm.

FIG. 4 shows representative images of B lineage cell expansion when plated as single cells. Single B cells from a different donor than in FIGS. 2 and 3 were expanded in conditions that included individual exogenously-added extracellular matrix proteins diluted in culture medium (Conditions 1, 2, and 3) and two pairwise combinations of exogenously-added extracellular matrix proteins (Conditions 4 and 5). Scale bars represent 200 μm.

FIG. 5 shows representative images of B lineage cell expansion when plated as single cells. Single B cells from the same donor in FIG. 4 were expanded in the presence of an individual exogenously-added extracellular matrix protein diluted in culture medium, and either in the presence or absence of a ligand of CD40. Circled regions highlight selected clusters of expanded cells. Scale bars represent 200 μm.

FIG. 6 shows representative images of B lineage cell expansion when plated as single cells. Single B cells from the same donor in FIG. 2 were expanded in the presence of individual exogenously-added extracellular matrix proteins (Conditions 1 and 2) diluted in culture medium, which were different from those tested in FIGS. 2-5. Scale bars represent 200 μm.

FIG. 7 shows representative images of B lineage cell expansion when plated as single cells. Single B cells from the same donor in FIG. 2 were expanded in the presence of a single exogenously-added extracellular matrix protein diluted in culture medium, but in the further presence or absence of different lipid mixes, as indicated. Scale bars represent 200 μm.

FIG. 8 shows a line graph of cell surface marker expression among B lineage cells expanded from a single B cell in the presence of two different, individual exogenously-added extracellular matrix proteins (Condition 1 and 2), and in combination (Condition 3). Bars represent the mean of 2 wells.

FIG. 9 shows representative images of B lineage cell expansion when plated as single cells. Single B cells were expanded in a culture medium comprising a single exogenously-added extracellular matrix protein, and different combinations of cytokines: IL-2 and IL-10 (Condition 1) and IL-2, IL-4, IL-6, IL-10, and IL-21 (Condition 2). Control conditions included: unsupplemented basal medium (Control 1); basal medium supplemented with cytokines but no exogenously-added extracellular matrix protein (Control 2); and in basal medium supplemented with extracellular matrix protein but no cytokines (Control 3). Scale bars represent 200 μm.

FIG. 10 shows representative images of B lineage cell expansion when plated as single cells. Single B cells were expanded in the presence of a single exogenously-added extracellular matrix protein diluted in different commercially available basal media, as indicated. White arrows indicate clusters of cells. Scale bars represent 200 μm.

FIG. 11 shows representative images of human memory B lineage cell expansion when plated as single cells. Single memory B cells from a new donor were expanded in conditions that included a combination of two (Condition 1) or a combination of three (Condition 2) extracellular matrix proteins diluted in a culture medium. Scale bars represent 200 μm.

FIG. 12 shows the results of expanding single human memory B cells according to this disclosure. Viable fold expansion after 12 days in culture is shown as mean±standard error of the mean (SEM) of 9 donors (A). IgM and IgG production by the expanded cells, as determined by ELISA, is shown as mean±SEM of 7 donors (B). Flow cytometry analysis of cell surface marker expression among day 12 expanded cells is shown as mean±SEM of 9 donors (C).

FIG. 13 shows the results of expanding single human plasmablasts according to this disclosure. Viable fold expansion after 12 days in culture is shown as mean±standard error of the mean (SEM) of 5 donors (A). IgM and IgG production by the expanded cells, as determined by ELISA, is shown as mean±SEM of 2 donors (B). Flow cytometry analysis of cell surface marker expression among day 12 expanded cells is shown as mean±SEM of 4 donors (C).

FIG. 14 shows representative images of mouse pan-B cell expansion when plated as single cells. Single pan-B cells from a C57 BL/6 mouse were expanded in the presence of two different combinations of two exogenously-added extracellular matrix proteins (Conditions 1 and 2) and in the presence of the indicated cytokine cocktail. Scale bars represent 200 μm.

FIG. 15 shows the results of expanding single mouse memory B cells according to this disclosure. Viable fold expansion after 12 days in culture is shown as mean±standard error of the mean (SEM) of 5 donors (A). IgG production by the expanded cells, as determined by ELISA, is shown as mean±SEM of 2 donors (B). Flow cytometry analysis of cell surface marker expression among day 12 expanded cells is shown as mean±SEM of 2-4 donors (C).

FIG. 16 shows the results of expanding single mouse plasmablasts according to this disclosure. Viable fold expansion after 12 days in culture is shown as mean±standard error of the mean (SEM) of 3 donors (A). IgG production by the expanded cells, as determined by ELISA, is shown as mean±SEM of 2 donors (B). Flow cytometry analysis of cell surface marker expression among day 12 expanded cells is shown as mean±SEM of 2-3 donors (C).

FIG. 17 shows the results of expanding T lineage cells plated as single cells. Viable fold expansion of single, mouse CD3⁺ T cells after 12 total days in culture is shown as mean±standard error of the mean (SEM) of 2 donors (A). Flow cytometry analysis of cell surface marker expression among day 12 expanded cells of (A) is shown as mean±SEM of 2-3 donors (B). Image of expanded human CD3⁺ T cells plated as single cells in the presence of a single exogenously-added extracellular matrix protein diluted in basal medium. Scale bars represent 200 μm (C).

### DETAILED DESCRIPTION

This disclosure relates to media compositions (and/or supplements to be added into a medium), and to methods for culturing lymphocytes, such as T cells, NK cells, or B cells. More specifically, this disclosure relates to expanding T cells, NK cells, or B cells that are seeded as single cells or at a clonal cell density into media and supplements as disclosed herein, and by practicing the methods disclosed herein.

Where used in this disclosure, the term “lymphocyte” refers to a cell of the lymphocyte lineage of the mammalian hematopoietic system, such as T cells, B cells, and/or NK cells. A mammalian lymphocyte may be a human, mouse, rat, or otherwise, lymphocyte. The lymphocyte may be derived from a stem cell, such as a hematopoietic stem cell (e.g. a CD34⁺ stem cell), or any other stem cell that may be obtained from cord blood, peripheral blood, or bone marrow. In one embodiment, the lymphocyte may be pluripotent-stem cell derived.

Where used in this disclosure “B cell” or “B cells” refers to a mammalian B lymphocyte or population of B lymphocytes. B cells may be comprised in a sample, and such sample may include one or more B cell populations, subpopulations, or subsets, including but not limited to, naïve B cells, memory B cells, activated B cells, B2 cells, B1 cells, germinal center B cells, marginal zone B cells, regulatory B cells, follicular B cells, isolated pan B cells, plasmablasts, plasma cells, and/or other B cell lineages. B cells may be distinguished from non B-cells, such as in a sample, on the basis of a biomarker such as a B-cell surface marker (e.g. an antigen expressed on the surface of a B cell that can be targeted with an agent that binds thereto). Exemplary B-cell surface markers include: CD10, CD19, CD20 (MS4A1), CD21, CD22, CD23, CD24, CD25, CD27, CD38, CD40, CD53, CD72, CD73, CD79a, CD79b, CD80, CD86, CD138, and TACI. Exemplary mouse B-cell surface markers include: CD19, CD20, CD22, CD27, CD28, CD38, CD44, CD80, CD73, CD86, CD93, CD98, CD138, CD45R, B220 and CD273.

Where used in this disclosure “T cell” or “T cells” refers to a mammalian T lymphocyte or population of T lymphocytes. T cells may be comprised in a sample, and such sample may include one or more T cell populations, subpopulations, or subsets, including but not limited to, pan-T cells, CD4⁺ T cells, CD8⁺ T cells, memory T cells, regulatory T cells, helper T cells, effector T cells, cytotoxic T cells, and/or naïve T cells, CAR-T cells, activated T cells, Th1 cells, Th2 cells, or other T cell lineages. T cells may be distinguished from non T-cells, such as in a sample, on the basis of a biomarker such as a T-cell surface marker (e.g. an antigen expressed on the surface of a T cell that can be targeted with an agent that binds thereto). T cells are typically characterized by: the absence of NK-, B-, and erythromyeloid-specific markers; the expression of CD3, and TCRαβ (or TCRγδ), and CD4 or CD8; and their effector functions. Although, some subsets of T cells may express characteristics of both T and NK cells, and may or may not express TCRαβ or TCRγδ and may or may not also express CD4, CD8, CD56, CD16 and NK1.1. T cells may further be characterized by the expression of CD8α, CD8β, CD45RA and CD27.

Where used in this disclosure “NK cell” or “NK cells” refers to a mammalian NK lymphocyte or population of NK lymphocytes. NK cells may be comprised in a sample, and such sample may include one or more NK cell populations, subpopulations, or subsets, including but not limited to, pan NK cells, tolerant NK cells, cytotoxic NK cells, regulatory NK cells, CAR-NK cells, or other NK cell lineages. NK cells may be distinguished from non NK-cells, such as in a sample, on the basis of a biomarker such as a NK-cell surface marker (e.g. an antigen expressed on the surface of a NK cell that can be targeted with an agent that binds thereto). NK cells are typically characterized by: the absence of T and B cell-specific markers; the expression of CD56 with or without CD16 (low affinity Fc gamma receptor 3A, expressed on a subset of NK cells); and their effector functions. NK cells may further be characterized by the expression of activating and inhibitory receptors referred to as killer immunoglobulin-like receptors (KIRs).

Where used in this disclosure, the term “extracellular matrix” refers to one or more molecules that provide structural and biochemical support to cells. Extracellular matrix compounds are also widely known to promote cell adhesion, cell-to-cell communication, and differentiation within a given tissue or agglomerate of cells in a culture medium. Both natural and synthetic extracellular matrices are contemplated within the present disclosure. Many different extracellular matrix proteins are known including, but not limited to: laminins, collagens, fibronectins, vitronectins, ECM1, osteopontins, fibronectins, and SPARC. An extracellular matrix may comprise one or more extracellular matrix proteins. Where an extracellular matrix comprises only one extracellular matrix protein, it may be said that it comprises one type of an extracellular matrix protein. Where an extracellular matrix comprises two different extracellular matrix protein, it may be said that it comprises two types of an extracellular matrix protein. Where an extracellular matrix comprises three different extracellular matrix protein, it may be said that it comprises three types of an extracellular matrix protein, and so on. Components of an extracellular matrix may include one or more of the following proteins: a fibronectin, a laminin, a vitronectin, a tenascin, an entactin, a thrombospondin, an elastin, a gelatin, a collagen, a fibrillin, a merosin, an anchorin, a chondronectin, a link protein, a bone sialoprotein, an osteocalcin, an osteopontin, an epinectin, a hyaluronectin, an undulin, an epiligrin, a kalinin, a synthetic polymer-based hydrogel (such as a polyethylene glycol, a polyvinyl, or any derivative or analogue thereof), and a plant-based hydrogel (such as a cellulose, a hemicellulose, a lignin, a starch and a pectin, or any derivative or analogue thereof). Extracellular matrix proteins of this disclosure may be comprised in a supplement to be added to a medium of this disclosure, or comprised in a medium of this disclosure. In both such embodiments, the extracellular matrix protein(s) may be soluble or solubilized.

Where used in this disclosure, the term “mixture of lipids” means a preparation of lipids and/or lipid-like substances. The mixture of lipids may be purchased from a commercial vendor or may be prepared just prior to use. Examples of lipids that may be comprised in the mixture of lipids include but are not limited to, propionic acid, butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, or octatriacontanoic acid, and/or from the group of unsaturated fatty acids comprising a-linolenic acid, stearidonic acid, eicosapentaenoic acid, docosahexaenoic acid, linoleic acid, γ-linolenic acid, dihomo-γ-linolenic acid, arachidonic acid, docosatetraenoic acid, palmitoleic acid, vaccenic acid, paullinic acid, oleic acid, elaidic acid, gondoic acid, erucic acid, nervonic acid, or mead acid. In one embodiment, the mixture of lipids may be comprised in a concentrated preparation, such as a supplement, to be added into a culture medium. In one embodiment, the mixture of lipids may already be included in a basal medium or a complete medium of this disclosure.

Where used in this disclosure, the term “cytokine” or “cytokines” refers to soluble proteins which are produced and released from a cell. Functions of cytokines include stimulating or repressing a cell of origion or other cells in direct or indirect contact. As an example, cytokines may mediate the generation or regulation of an immune response. Common cytokines include: interleukins IL-1 through IL-15; tumor necrosis factors α& β; interferons α, β and γ; TGF-β; colony stimulating factor (CSF); and granulocyte monocyte colony stimulating factor (GM-CSF). One or more cytokines may be included in media or supplements of this disclosure, or used in methods to expand lymphocytes, such as T, NK, or B cells.

Where used in the disclosure, the term “clonal cell density” or “clonal density” refers to a density at which a cell or cells are capable of forming individual colonies or clusters. In a particular embodiment, the cells are seeded at a density of 1 cell/well. In an embodiment, the cells are seeded at a density of at least 1 cell/well. In one embodiment, the cells may be seeded at a density of 1000 cells/well in a culture dish, for example, a 10 cm culture dish. In an embodiment, the cells are seeded at a sufficiently low density to result in the effective isolation of single, non-impinging cells when plated in a culture dish/well.

### Media and Supplements

In one aspect of this disclosure are provided media for expanding mammalian lymphocytes, such as T, B, or NK cells. In one aspect of this disclosure are provided cell culture media supplements to be added to media for expanding mammalian lymphocytes, such as T, B, or NK cells, or progenitors thereof. Both the media and the supplements may include factors and other components important for expanding lymphocytes, such as T, B, or NK cells, or progenitors thereof, whether or not plated as single cells or at a clonal cell density. In some embodiments, the media and supplements may also include additional factors and other components that improve the efficiency of lymphocyte or progenitor expansion and/or differentiation and/or activation, whether or not plated as single cells or at a clonal cell density.

In one embodiment, the mammalian B cells are human B cells. In one embodiment, the human B cells are memory B cells. In one embodiment, the human B cells are naïve B cells. In one embodiment, the human B cells are pan B cells. In one embodiment, the mammalian B cells are plasmablasts. In one embodiment, the mammalian B cells are plasma cells. In one embodiment, the mammalian B cells are mouse B cells. However, one of ordinary skill in the art will readily appreciate that the cell culture media supplements of the disclosure may be applied to other cell types, such as T cells or NK cells.

Culture media of this disclosure will comprise a basal medium. The basal medium may be any medium, when appropriately supplemented, capable of supporting the expansion of mammalian lymphocytes or progenitors thereof, such as T or B cells. In one embodiment, the basal medium may be any medium, when appropriately supplemented, capable of supporting the expansion of human mammalian lymphocytes or progenitors thereof, such as T or B cells. In one embodiment, the basal medium may be any medium, when appropriately supplemented, capable of supporting the expansion of rodent (e.g. mouse) mammalian lymphocytes or progenitors thereof, such as T or B cells. In one embodiment, the basal medium may be any medium capable of supporting the expansion of human or mouse memory B cells or plasmablasts.

Numerous basal media are known in the art, and many of which are commercially available. Examples of basal media may include RPMI, DMEM/F-12, IMDM, StemSpan™ and ImmunoCult™ branded media. Basal medium typically include one or more of carbohydrates, amino acids, trace elements, lipids, buffers, salts, proteins including albumin, and the like. In some embodiments, basal media may not include one or more of the foregoing types of components, and may be correspondingly supplemented when formulating a complete medium.

A basal medium used to formulate mammalian lymphocyte expansion media of this disclosure may be supplemented with additional components to make a complete medium. In one embodiment, a mammalian lymphocyte expansion media of this disclosure may be provided as a complete medium (i.e. requires no further supplementation). In one embodiment, a basal medium may be provided together (whether in a kit or otherwise) with one or more supplements to be added to the basal medium to formulate a complete medium prior to use.

To formulate complete mammalian lymphocyte expansion media, it may be beneficial and/or necessary to include (or further supplement with) one or more of: growth factor(s), extracellular matrix protein(s), such as cytokine(s), lipid(s), hormone(s), salt(s), vitamin(s), other proteins including albumin(s), small molecule(s), etc.

In one embodiment, culture media (e.g. mammalian lymphocyte expansion media) of this disclosure comprise a basal medium, and one or more of a mixture of lipids, a ligand of CD40, and one or more cytokines. In one embodiment, culture media (e.g. mammalian lymphocyte expansion media) of this disclosure comprise a basal medium, and two or more of a mixture of lipids, a ligand of CD40, and one or more cytokines. In one embodiment, culture media (e.g. mammalian lymphocyte expansion media) of this disclosure comprise a basal medium, and each of a mixture of lipids, a ligand of CD40, and one or more cytokines.

In one embodiment, a mixture of lipids may include a single type of lipid. In one embodiment, the mixture of lipids may include more than one type of lipids. In one embodiment, a mixture of lipids may be comprised in a basal medium. In one embodiment, a mixture of lipids may be comprised in a supplement to be added to a basal medium.

In one embodiment, the mixture of lipids is chemically defined. In one embodiment, the mixture of lipids may be animal component free.

Culture media of this disclosure may comprise one or more cytokine and/or one or more growth factor. In one embodiment, the type(s) of the one or more cytokines and/or the one or more growth factors comprised in cell culture media of this disclosure are selected depending on the specific type of cell to be cultured, such as a lymphocyte (e.g. a T, NK or B cell).

In one embodiment, the one or more cytokine or growth factor may be any one or combination of IL-2, IL-4, IL-6, IL-10, and IL-21. In one embodiment, mammalian lymphocyte expansion media are supplemented with each of IL-2, IL-4, IL-6, IL-10, and IL-21. In one embodiment, mammalian lymphocyte expansion media are supplemented with one or more of IL-2, IL-4, IL-6, IL-10, and IL-21. In one embodiment, mammalian lymphocyte expansion media are supplemented with at least IL-2 and IL-10, and optionally IL-21.

In specific embodiments, mammalian B cell or T cell expansion media comprise each of IL-2, IL-4, IL-6, IL-10, and IL-21. In other specific embodiments, mammalian B cell or T cell expansion media comprise at least IL-2 and IL-10, and optionally IL-21.

One or more cytokines comprised in culture media of this disclosure, may respectively be present at concentrations between about 10 ng/ml-200 μg/mL, or about 0.1-100 μg/mL, or about 1-50 μg/mL, or about 5-25 μg/mL. In embodiments where small molecule analogues of one or more of the foregoing cytokines are included in mammalian lymphocyte expansion media, they are typically used at lower concentrations.

Culture media of this disclosure may comprise a ligand of CD40. In one embodiment, a ligand of CD40 binds to a CD40L receptor to promote activation of the cell. In one embodiment, the ligand of CD40 is a ligand of human CD40. In one embodiment, the ligand of CD40 comprises one or more proteins. In one embodiment, the ligand of CD40 is comprised in a complex of proteins. In one embodiment, the concentration of the ligand of CD40L in the culture media ranges between about 0.1 ng/ml to 500 ng/ml, about 0.5 ng/ml to 100 ng/ml, or about 1 ng/ml to 50 ng/ml.

Culture media of this disclosure may comprise a first type of an extracellular matrix protein. In one embodiment, culture media of this disclosure comprise a first type and a second type of an extracellular matrix protein. In one embodiment, culture media of this disclosure comprise a first type of an extracellular matrix protein, a second type of an extracellular matrix protein, and a third type of an extracellular matrix protein. In some embodiments, culture media of this disclosure comprise more than three types of extracellular matrix proteins. In one embodiment, the first type of an extracellular matrix protein may be selected from a collagen, an ECM1, a laminin, an osteopontin, a vitronectin, or a SPARC. In embodiments including a second type, a third type, or further types of an extracellular matrix protein, they may be selected from, but not limited to, a collagen, an ECM1, an E-Cadherin, a laminin (e.g., laminin-511, a fragment of laminin-511 (e.g., laminin 511-E8), laminin-521 (also known as laminin 11), an osteopontin, a fibronectin, a vitronectin, a SPARC or a combination thereof. In a preferred embodiment, the extracellular matrix protein(s) comprised in a culture medium of this disclosure are one or more of an ECM1, an osteopontin, a vitronectin, a collagen, and a laminin, and more preferably one or more of an ECM1, an osteopontin, and a vitronectin.

In one embodiment, a concentration of an extracellular matrix protein (whether the first type, the second type, the third type, or further types) ranges between about 0.1 μg/ml to 1 mg/mL. In one embodiment, a concentration of an extracellular matrix protein (whether the first type, the second type, the third type, or further types) ranges between about 1 μg/ml to 100 μg/mL, about 3 μg/mL to 50 μg/mL, about 5 μg/ml to 30 μg/mL or about 10 g/mL to 20 μg/mL.

In one embodiment, the first type of an extracellular matrix protein, the second type of an extracellular matrix protein, or the third type of an extracellular matrix protein is coated or immobilized on the surface of a particle/bead or culture receptacle/vessel in which the lymphocytes (e.g. B or T cell(s)) are contained. Thus, in such an embodiment, a cell cultured in the receptacle is cultured in the presence of a medium of this disclosure and in the presence of a first type of an extracellular matrix protein (and any further types of extracellular matrix proteins) coated on or bound to a particle/bead or surface of the cell culture receptacle/vessel.

In one embodiment, each of the extracellular matrix proteins included in the culture medium (or, on a particle or receptacle) is recombinant. In one embodiment, some but not all of the extracellular matrix proteins are recombinant. In one embodiment, the one or more extracellular matrix proteins are recombinant human extracellular matrix proteins. In one embodiment, the one or more extracellular matrix proteins are recombinant mouse extracellular matrix proteins.

In one embodiment, one or more of the extracellular matrix proteins (such as may be included in a culture medium of this disclosure) may be of natural origin, and purified from human, animal, or plant tissue. In one embodiment, the extracellular matrix protein(s) may be genetically engineered and/or recombinant proteins, or otherwise synthetic in nature. In one embodiment, the extracellular matrix protein(s) may be a whole protein or a fragment thereof, such as a peptide fragment.

In one embodiment, culture media of this disclosure expand mammalian lymphocytes or progenitors thereof plated as single cells. Expansion of cells having been plated as single cells may be important when a particular cell is intended to be cloned, such as a cell having had its genome edited.

In one embodiment, culture media of this disclosure expand mammalian lymphocytes or progenitors thereof plated at a clonal density. Cells may be plated at a clonal density in a microplate, and in applicable embodiments it is important that a cell density is chosen so that the cells are sufficiently spaced apart. Cells plated at a clonal density may have a low probability of coming into contact with one another. Cells plated at a clonal cell density may also not impart paracrine effects upon other cells in the container. For example, a clonal density may correspond to a cell density of 1 cell/cm², 10 cells/cm², 100 cells/cm², or 1000 cells/cm².

In one embodiment, culture media of this disclosure expand mammalian lymphocytes or progenitors thereof not plated at a clonal density.

In one embodiment, the lymphocytes or progenitors thereof expanded using the foregoing supplements and/or media formulations may be used in higher compliance applications, such as for cell therapy. In one embodiment, media of this disclosure are chemically defined. In one embodiment, media of this disclosure may be serum-free and/or animal component-free. Such media may nevertheless include an albumin, such as a recombinant albumin. Thus, media of this disclosure may support serum-and/or animal component-free expansion of lymphocytes or progenitors thereof.

In one embodiment, lymphocytes or progenitors thereof cultured in media of this disclosure do not come into contact with feeder cells or undefined cell supports (e.g. matrices), such as Matrigel™. Thus, media of this disclosure may support feeder-free expansion of lymphocytes or progenitors thereof.

Overall, media of this disclosure, whether provided as a complete medium or as a basal medium to be supplemented as described herein, will support the expansion of mammalian lymphocytes. In embodiments where the lymphocytes are B cells, they may be pan-B cells, memory B cells, naïve B cells, plasmablasts, plasma cells, or other B cell lineages. In embodiments where the lymphocytes are T cells, they may be pan-T cells, CD4⁺ T cells, CD8⁺ T cells, memory T cells, regulatory T cells, helper T cells, effector T cells, cytotoxic T cells, and/or naïve T cells, CAR-T cells, or other T cell lineages. In embodiments where the lymphocytes are NK cells, they may be pan NK cells, cytotoxic NK cells, regulatory NK cells, tolerant NK cells, CAR-NK cells.

In one embodiment, mammalian lymphocyte expansion media of this disclosure support 5 or more, 10 or more, 15 or more, 20 or more, 25 or more, 30 or more, 35 or more, 40 or more, 45 or more, 50 or more, 55 or more, or 60 or more population doublings.

In one embodiment, mammalian lymphocytes or progenitors thereof expanded using media of this disclosure express markers characteristic of mammalian B, NK or T cell lineages. In one embodiment, more than 30% of the expanded cells are positive for a marker characteristic of mammalian B, NK or T cell lineages. In one embodiment, more than 50% of the expanded cells are positive for a marker characteristic of mammalian B, NK or T cell lineages. In one embodiment, more than 60% of the expanded cells are positive for a marker characteristic of mammalian B, NK or T cell lineages. In one embodiment, more than 70% of the expanded cells are positive for a marker characteristic of mammalian B, NK or T cell lineages.

In one embodiment, media of this disclosure may further support differentiation of the lymphocytes or progenitors thereof in the culture environment. In one embodiment, media support 10% or more of the B, NK or T cells, or progenitors thereof, to differentiate/mature. In one embodiment, media support 20% or more of the B, NK or T cells, or progenitors thereof, to differentiate/mature. In one embodiment, media support 30% or more of the B, NK or T cells, or progenitors thereof, to differentiate/mature. In one embodiment, media support 40% or more of the B, NK or T cells, or progenitors thereof, to differentiate/mature. In one embodiment, media support 50% or more of the B, NK or T cells, or progenitors thereof, to differentiate/mature. In one embodiment, media support 60% or more of the B, NK or T cells, or progenitors thereof, to differentiate/mature.

In one embodiment, media of this disclosure may further support activation of the lymphocytes or progenitors thereof in the culture environment.

In another aspect of this disclosure is provided a cell culture media supplement for expanding mammalian lymphocytes or progenitors thereof. In one embodiment, the mammalian lymphocytes are plated as single cells or at a clonal cell density. In one embodiment, the mammalian lymphocytes are not plated as single cells or at a clonal cell density.

In one embodiment, the cell culture media supplement may comprise one or more of: a ligand of CD40; a mixture of lipids; and one or more cytokines. In one embodiment, the cell culture media supplement may comprise a diluent and one or more of: a ligand of CD40, a mixture of lipids, and one or more cytokines. In one embodiment, the cell culture media supplement may comprise two or more of: a ligand of CD40; a mixture of lipids; and one or more cytokines. In one embodiment, the cell culture media supplement may comprise a diluent and two or more of: a ligand of CD40, a mixture of lipids, and one or more cytokines. In one embodiment, the cell culture media supplement may comprise each of: a ligand of CD40; a mixture of lipids; and one or more cytokines. In one embodiment, the cell culture media supplement may comprise a diluent and each of: a ligand of CD40, a mixture of lipids, and one or more cytokines.

In one embodiment the diluent is physiological, such as in terms of pH, salt concentration, osmolality, osmolarity, and the like. In one embodiment, the diluent is inorganic. By way of non-limiting example, the diluent may be aqueous (e.g. water). In one embodiment, the diluent may be a buffer. In one embodiment, the diluent solubilizes the components therein, such one or two or more of a ligand of CD40; a mixture of lipids; and one or more cytokines. In one embodiment, the diluent solubilizes extracellular matrix protein(s) that may be comprised in the supplement.

In one embodiment, a supplement of this disclosure comprises a mixture of lipids. The above description regarding a mixture of lipids and its concentration in media may apply to supplements of this disclosure. However, in embodiments where a supplement is concentrated (e.g. >1×), then the concentration ranges will be appropriately adjusted.

In one embodiment, a supplement of this disclosure comprises a ligand of CD40. The above description regarding CD40 ligands and its concentration in media may apply to supplements of this disclosure. However, in embodiments where a supplement is concentrated (e.g. >1×), then the concentration ranges will be appropriately adjusted.

In one embodiment, a supplement of this disclosure comprises one or more cytokine and/or one or more growth factor. The above description regarding cytokines and their concentration in media may apply to supplements of this disclosure. However, in embodiments where a supplement is concentrated (e.g. >1×), then the concentration ranges will be appropriately adjusted.

In one embodiment, the supplement may comprise at least a first type of an extracellular matrix protein and one or more of: a ligand of CD40; a mixture of lipids; and one or more cytokines. In one embodiment, the supplement may comprise at least a first type of an extracellular matrix protein and two or more of: a ligand of CD40; a mixture of lipids; and one or more cytokines. In one embodiment, the supplement may comprise at least a first type of an extracellular matrix protein and all of: a ligand of CD40, a mixture of lipids, and one or more cytokine.

In one embodiment, the supplement comprises a first type and a second type of an extracellular matrix protein. In one embodiment, the supplement comprises a first type of an extracellular matrix protein, a second type of an extracellular matrix protein, and a third type of an extracellular matrix protein. In some embodiments, supplements of this disclosure comprise more than three types of extracellular matrix proteins. In one embodiment, at least one extracellular matrix protein is contained in a supplement used to formulate a complete expansion medium of this disclosure.

In embodiments of a supplement of this disclosure, the above description of one, two or three, or more types of extracellular matrix proteins and their concentration(s) in media may apply to supplements of this disclosure. However, in embodiments where a supplement is concentrated (e.g. >1×), then the concentration ranges will be appropriately adjusted.

By way of example, the one, two, three, or more types of an extracellular matrix protein comprised in a supplement may be selected from a collagen, an ECM1, a laminin, an osteopontin, a vitronectin, or a SPARC. In embodiments including a second type, a third type, or further types of an extracellular matrix protein, they may be selected from, but not limited to, a collagen, an ECM1, an E-Cadherin, a laminin (e.g., laminin-511, a fragment of laminin-511 (e.g., laminin 511-E8), laminin-521 (also known as laminin 11)), an osteopontin, a fibronectin, a vitronectin, a SPARC or a combination thereof. In a preferred embodiment, the extracellular matrix protein(s) comprised in a supplement of this disclosure are one or more of an ECM1, an osteopontin, a vitronectin, a collagen, and a laminin, and more preferably one or more of an ECM1, an osteopontin, and a vitronectin.

In one embodiment, a supplement of this disclosure may be concentrated. In one embodiment, the supplement is at about a 2× concentration, about a 5× concentration, about a 10× concentration, about a 15× concentration, about a 20× concentration, about a 25× concentration, about a 50× concentration, about a 100× concentration, about a 200× concentration, about a 500× concentration, or greater.

In one embodiment, supplements of this disclosure (when formulated in a mammalian lymphocyte expansion media) support 5 or more, 10 or more, 15 or more, 20 or more, 25 or more, 30 or more, 35 or more, 40 or more, 45 or more, 50 or more, 55 or more, or 60 or more population doublings.

In one embodiment, supplements of this disclosure (when formulated in a mammalian lymphocyte expansion media) may further support differentiation/maturation of the lymphocytes or progenitors thereof (e.g from a more primitive to a more specified fate) in the culture environment. In one embodiment, 10% or more of the B, NK or T cells, or progenitors thereof, differentiate/mature. In one embodiment, 20% or more of the B, NK or T cells, or progenitors thereof, differentiate/mature. In one embodiment, 30% or more of the B, NK or T cells, or progenitors thereof, differentiate/mature. In one embodiment, 40% or more of the B, NK or T cells, or progenitors thereof, differentiate/mature. In one embodiment, 50% or more of the B, NK or T cells, or progenitors thereof, differentiate/mature.

In one embodiment, supplements of this disclosure (when formulated in a mammalian lymphocyte expansion media) may further support activation of the lymphocytes or progenitors thereof in the culture environment.

In one embodiment, a supplement (and media combined with the supplement) of this disclosure does not, and the cells cultured in the presence thereof do not, come into contact with feeder cells or undefined cell supports (e.g. matrices), such as Matrigel™. Thus, a supplement (and media combined with the supplement) of this disclosure may support feeder-free expansion of lymphocytes (e.g. B or T cells) or progenitors thereof.

In one embodiment, a supplement (and media combined with the supplement) of this disclosure is animal component-free. Thus, a supplement (and media combined with the supplement) of this disclosure may support animal component-free expansion of lymphocytes (e.g. B or T cells) or progenitors thereof.

In one embodiment, a supplement (and media combined with the supplement) of this disclosure may be serum-free. Serum-free supplements (and media combined with the supplement) of this disclosure may nevertheless include an albumin, such as a recombinant albumin.

### Methods

In another aspect of this disclosure are provided methods of expanding mammalian lymphocytes, such as B cells or T cells, or progenitors thereof. In one embodiment, the mammalian lymphocytes are plated as single cells or at a clonal density. In one embodiment, the mammalian lymphocytes are not plated as single cells or at a clonal density.

The lymphocytes that are seeded/cultured in the methods described herein may be isolated/enriched from a mixed sample, such as a blood or leukapheresis sample. In embodiments where the lymphocytes are B cells, the methods may involve seeding pan B cells, memory B cells, naïve B cells, plasmablasts, and/or plasma cells. In embodiments where the lymphocytes are T cells, the methods may involve seeding pan T cells, CD4⁺ T cells, CD8⁺ T cells, memory T cells, regulatory T cells, helper T cells, effector T cells, cytotoxic T cells, naïve T cells, CAR-T cells. In embodiments, where the lymphocytes are NK cells, the methods may involve seeded pan NK cells, cytotoxic NK cells, regulatory NK cells, tolerant NK cells, or CAR-NK cells. Notwithstanding, one of ordinary skill in the art will readily appreciate that the media/supplements and methods of the disclosure may be used with other subsets or populations of lymphocytes.

In one embodiment, the mammalian lymphocytes or progenitors thereof are human. In one embodiment, the mammalian lymphocytes or progenitors thereof are rodent (e.g. mouse).

In one embodiment, methods of expanding mammalian lymphocytes or progenitors thereof comprise seeding the lymphocytes (such as a population of B cells or T cells, or progenitors thereof) as single cells or at a clonal density into a culture environment. In one embodiment, methods of expanding mammalian lymphocytes or progenitors thereof comprise seeding the lymphocytes (such as a population of B, NK or T cells, or progenitors thereof) in bulk, and not as single cells or at a clonal density, into a culture environment. After seeding the cells, the methods may comprise incubating (e.g. culturing) the seeded cells or progenitors thereof in the culture environment for a time sufficient to expand the lymphocytes or progenitors thereof to a desired quantity.

In one embodiment, the mammalian lymphocytes or progenitors thereof are seeded into a culture flask, dish, vessel, plate, or a well thereof. Plating the lymphocytes may be important in cloning operations, such as when their genome has been edited or if the cell possesses a desired phenotype or performs a desired a function. In embodiments where mammalian B cells or progenitors thereof are plated at a clonal density, it is important that a cell density is chosen so that the cells are sufficiently spaced apart. Cells plated at a clonal density may have a low probability of coming into contact with one another. Desirably, cells plated at a clonal cell density do not impart paracrine effects upon other cells in the container. For example, a clonal density may correspond to a cell density of between 1 cell/well in a 6 well, 24 well, 48 well or a 96 well plate. In one embodiment, the cells may be seeded at a density of up to 10, 100, 1000 cells/well or cm² in a culture dish, for example, a 10 cm culture dish. As stated hereinabove, in some applications the cells are not plated as single cells or at a clonal cell density. For example, the cells may be plated at a density that is greater than 1000 cells/well or cm².

In one embodiment of the methods disclosed herein, the seeded cells are incubated in the culture environment for any time that is sufficient to expand the lymphocytes or progenitors thereof to a desired number. In one embodiment, the incubation step may be at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, or at least 14 days, or longer.

In one embodiment, the incubation step may be split into stages. For example, a first stage of incubation/expansion may be between 1-10 days in a first culture environment (e.g. culture medium formulation). In one embodiment, a first stage of incubation/expansion is between about 1-5 days, or between about 3-10 days, in a first culture environment (e.g. culture medium formulation). In one embodiment, a second stage of incubation/expansion is between about 1-10 days in a second culture environment (e.g. culture medium formulation). In one embodiment, a second stage of incubation/expansion is between about 1-5 days, or between about 3-10 days, in a second culture environment (e.g. culture medium formulation).

The culture environment into which the lymphocytes are seeded (and in which the lymphocytes are incubated) may comprise one or more of a culture medium, a container, a temperature and atmospheric conditions, etc.

With regard to culture medium (and supplements to be added to a medium to make a complete medium) the description above applies. For example, the culture environment (e.g. a culture medium) may comprise one or more of a mixture of lipids, a ligand of CD40, and one or more cytokines. In one embodiment, the culture environment (e.g. a culture medium) may comprise two or more of a mixture of lipids, a ligand of CD40, and one or more cytokines. In one embodiment, the culture environment (e.g. a culture medium) may comprise each of a mixture of lipids, a ligand of CD40, and one or more cytokines.

In one embodiment, a mixture of lipids comprises a single type of lipid. In one embodiment, a mixture of lipids comprises more than one type of lipid. In one embodiment, a mixture of lipids may be stabilized and/or animal component free.

In one embodiment, a ligand of CD40 is a ligand of human CD40. In one embodiment, a ligand of CD40 is a ligand of mouse or rat CD40. In one embodiment, a ligand of CD40 comprises one or more proteins. In one embodiment, a ligand of CD40 is comprised in a complex of proteins.

In one embodiment, a ligand of CD40 is contained in the culture medium. In one embodiment, a ligand of CD40 is otherwise included in the culture environment, such as coated on a surface of a container.

As described hereinabove, the type(s) of the one or more cytokines and/or the one or more growth factors comprised in a cell culture environment (e.g. cell culture media) of this disclosure are selected depending on the specific type of cell to be cultured.

In one embodiment, the one or more cytokine or growth factor may be any one or combination of IL-2, IL-4, IL-6, IL-10, and IL-21. In one embodiment, the one or more cytokine or growth factor comprises each of IL-2, IL-4, IL-6, IL-10, and IL-21. In one embodiment, the one or more cytokine or growth factor comprises at least IL-2 and IL-10, and optionally IL-21.

In one embodiment, the one or more cytokine and/or one or more growth factor is contained in a culture medium. In one embodiment, the one or more cytokine and/or one or more growth factor is otherwise included in the culture environment, such as coated on a surface of a container.

In one embodiment, the culture environment comprises a container (as described above) containing a culture medium (as described in detail herein). In one embodiment, the culture environment may further comprise a coating on a surface of the container. In one embodiment, the culture environment does not comprise a coating on a surface of the container.

In one embodiment, the culture environment (e.g. a culture medium) may further comprise a first type of an extracellular matrix protein. The first type of extracellular matrix protein may be comprised in a culture medium (or a supplement to be added thereto). In one embodiment, the first type of extracellular matrix protein may be solubilized in a culture medium (or a supplement to be added thereto).

In one embodiment, the first type of an extracellular matrix proteins may be selected from a collagen, an ECM1, a laminin, an osteopontin, a vitronectin, or a SPARC. In one embodiment, the first type of an extracellular matrix protein is ECM1, vitronectin, or osteopontin.

In one embodiment, the culture environment (e.g. a culture medium) may further comprise a second type of an extracellular matrix protein. The second type of extracellular matrix protein may be comprised in a culture medium (or a supplement to be added thereto). In one embodiment, the second type of extracellular matrix protein may be solubilized in a culture medium (or a supplement to be added thereto).

In one embodiment, the second type of an extracellular matrix proteins is different from the first type of an extracellular matrix protein and may be selected from a collagen, an ECM1, a laminin, an osteopontin, a vitronectin, or a SPARC. In one embodiment, the second type of an extracellular matrix protein is ECM1, vitronectin, or osteopontin.

In one embodiment, the culture environment (e.g. a culture medium) may further comprise a third type of an extracellular matrix protein. The third type of extracellular matrix protein may be comprised in a culture medium (or a supplement to be added thereto). In one embodiment, the third type of extracellular matrix protein may be solubilized in a culture medium (or a supplement to be added thereto).

In one embodiment, the third type of an extracellular matrix proteins is different from the first and second type of an extracellular matrix protein and may be selected from a collagen, an ECM1, a laminin, an osteopontin, a vitronectin, or a SPARC. In one embodiment, the third type of an extracellular matrix protein is ECM1, vitronectin, or osteopontin.

In one embodiment the second and third type of an extracellular matrix protein may be selected from a collagen, an ECM1, an E-Cadherin, a laminin (e.g., laminin-511, a fragment of laminin-511 (e.g., laminin 511-E8), laminin-521 (also known as laminin 11)), an osteopontin, a fibronectin, a vitronectin, a SPARC or a combination thereof.

Thus, in embodiments of the methods where the culture environment comprises a culture medium, at least a first type of an extracellular matrix protein, and one or more of a mixture of lipids, a ligand of CD40, and one or more cytokines may be comprised in the culture medium. If additional extracellular matrix proteins (e.g second, third, and further) are included in the culture environment, such extracellular matrix protein(s) may also be comprised in the culture medium.

In one embodiment, the culture environment (e.g. the medium or the supplement added thereto) comprises at least a first type of an extracellular matrix protein, optionally a second type of an extracellular matrix protein, and only one of a mixture of lipids, a ligand of CD40, and one or more cytokines. In one embodiment, the culture environment (e.g. the medium or the supplement added thereto) comprises at least a first type of an extracellular matrix protein, optionally a second type of an extracellular matrix protein, and two of a mixture of lipids, a ligand of CD40, and one or more cytokines.

The incubating/culturing step may be performed in different or in staged culture environments, for example comprising a first stage or culture environment and a second stage or a culture environment. In one embodiment, a first stage of incubating/culturing may be performed in a first culture environment, wherein such first culture environment comprises at least a first type of an extracellular matrix protein, and a second stage of incubating/culturing may be performed in a second culture environment, wherein such second culture environment does not comprise at least a first type of an extracellular matrix protein. In one embodiment, a first stage of incubating/culturing may be performed in a first culture environment, wherein such first culture environment does not comprise at least a first type of an extracellular matrix protein, and a second stage of incubating/culturing may be performed in a second culture environment, wherein such second culture environment comprises at least a first type of an extracellular matrix protein.

It should be noted that the first and second (or additional) stages or culture environments, may differ from one another in regard to any culture environment component/condition disclosed herein. For example, the stages or environments may differ in regard to: i) a mixture of lipids; ii) a ligand of CD40; iii) the one or more cytokines; iv) not a first type of an extracellular matrix protein, but a second and/or third type of an extracellular matrix protein; v) a coating of a container; and/or vi) their duration.

In one embodiment the culture environment further comprises a coating or functionalization applied to a surface of a container. In embodiments where the culture environment comprises both a culture medium and a coating/functionalization applied to a surface of a container, the coating on the surface of the container may comprise one or more of a first type of an extracellular matrix protein, a second type of an extracellular matrix protein, and a third type of an extracellular matrix protein. In such embodiments, the culture medium may also comprise one or more types of extracellular matrix proteins.

In one embodiment, lymphocytes (e.g. B, NK or T cells) or progenitors thereof cultured using the methods of this disclosure do not come into contact with feeder cells or undefined cell supports (e.g. matrices), such as Matrigel™. Thus, methods of this disclosure may support feeder-free expansion of lymphocytes or progenitors thereof.

In one embodiment, lymphocytes (e.g. B, NK or T cells) or progenitors thereof cultured using the methods of this disclosure do not come into contact with animal-derived components. Thus, methods of this disclosure may support animal component-free expansion of lymphocytes or progenitors thereof. In one embodiment, an animal component-free workflow may be a serum-free workflow.

Overall, the practice of the methods (and use of the media) of this disclosure may yield an expanded population of mammalian lymphocytes (e.g. B, NK or T cells) or progenitors thereof. More particularly, the practice of the methods (and use of the media) of this disclosure may yield up to a 1000-fold, or up to 5000-fold, or up to a 10,000-fold, or up to a 15,000-fold, or up to a 20,000-fold, or up to a 25,000-fold or higher increase in mammalian lymphocytes (e.g. B, NK or T cells) or progenitors thereof.

In one embodiment, methods of this disclosure support 5 or more, 10 or more, 15 or more, 20 or more, 25 or more, 30 or more, 35 or more, 40 or more, 45 or more, 50 or more, 55 or more, or 60 or more population doublings.

In one embodiment, mammalian lymphocytes (e.g. T, NK or B cells) or progenitors thereof expanded using the methods of this disclosure express markers characteristic of the expanded lymphocyte lineage. In one embodiment, more than 30% of the expanded cells are positive for one or more markers characteristic of mammalian B, NK and/or T cell lineages. In one embodiment, more than 40% of the expanded cells are positive for one or more markers characteristic of mammalian B, NK and/or T cell lineages. In one embodiment, more than 50% of the expanded cells are positive for one or more markers characteristic of mammalian B, NK and/or T cell lineages. In one embodiment, more than 60% of the expanded cells are positive for one or more markers characteristic of mammalian B, NK and/or T cell lineages. In one embodiment, more than 70% of the expanded cells are positive for one or more markers characteristic of mammalian B, NK or T cell lineages.

In one embodiment, methods of this disclosure may further support differentiation/maturation of the lymphocytes or progenitors thereof (e.g from a more primitive to a more specified fate) in the culture environment, such as during expansion. In one embodiment, 10% or more of the B, NK or T cells, or progenitors thereof, differentiate/mature. In one embodiment, 20% or more of the B, NK or T cells, or progenitors thereof, differentiate/mature. In one embodiment, 30% or more of the B, NK or T cells, or progenitors thereof, differentiate/mature. In one embodiment, 40% or more of the B, NK or T cells, or progenitors thereof, differentiate/mature. In one embodiment, 50% or more of the B, NK or T cells, or progenitors thereof, differentiate/mature.

In one embodiment, methods of this disclosure (that may support differentiation of the lymphocytes (e.g. B or T cells) or progenitors thereof in the culture environment) involve an incubating step. In one embodiment, the incubating step is 1-15 days. In one embodiment, the incubating step is more than 1 day. In one embodiment, the incubating step is at least 3 days.

In one embodiment, methods of this disclosure may further support activation of the lymphocytes or progenitors thereof in the culture environment (e.g. while incubating in the culture medium), such as during expansion and/or differentiation.

The following non-limiting examples are illustrative of the present disclosure.

### EXAMPLES

### Example 1: Processing Blood Samples to Enrich for B Lineage Cells

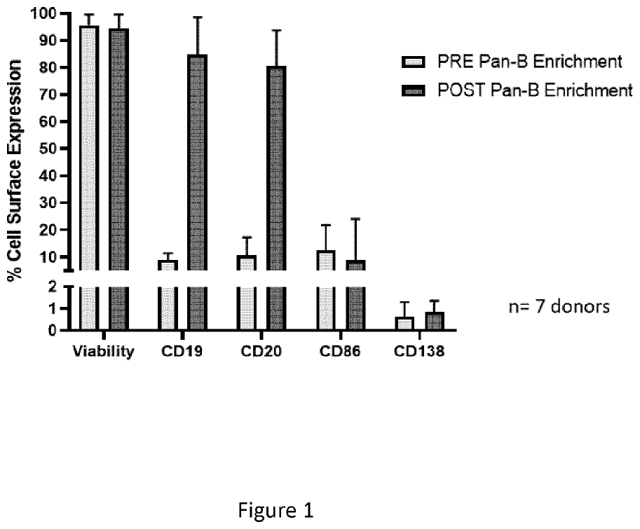

Pan-B cells were enriched from human peripheral blood mononuclear cells (PBMCs, i.e. leukapheresis samples) using EasySep™ Human Pan-B Cell Enrichment Kit (STEMCELL Technologies). The cells of leukapheresis samples and the B lineage cells enriched therefrom were assessed for viability by flow cytometry analysis after staining with the fluorescent DRAQ7 dye (FIG. 1). Also, the B lineage cells enriched from leukapheresis samples were assessed for purity and activation status by flow cytometry using fluorescently labelled antibodies to the surface markers CD19, CD20, CD86, and CD138 (FIG. 1).

### Example 2: Seeding and Culturing Enriched B Lineage Cells

Pan-B cells were enriched as described in Example 1, and single pan-B cells were sorted into respective wells of a 96-well plate (BD FACSAria™ Fusion Cell Sorter). Unless indicated otherwise, each well included ImmunoCult™-XF B Cell Base Medium (STEMCELL Technologies) that was supplemented with a ligand of human CD40 (various suppliers, including STEMCELL Technologies), one or more extracellular matrix proteins (STEMCELL Technologies, and other commercial sources), a mixture of lipids (STEMCELL Technologies), and one or more cytokines (STEMCELL Technologies). Those extracellular matrix proteins shown in this study to expand single cells include one or more of a collagen, an ECM1, an E-Cadherin, a laminin, an osteopontin, a fibronectin, a vitronectin, or a SPARC, and preferably one or more of a collagen, an ECM1, a laminin, an osteopontin, and a vitronectin. Wells were imaged with an Olympus Q Colour 3 microscope at 40× periodically during an expansion protocol.

### Example 3: Single B Cells Markedly Expand in the Presence of Soluble, Exogenously-Added Extracellular Matrix Protein(s)

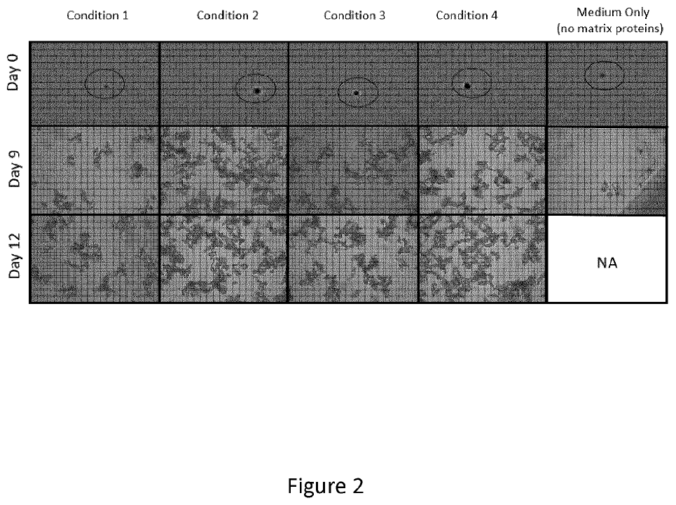

Pan-B cells were enriched and seeded essentially as described in Examples 1 and 2, and the plating of a single pan-B cell per well at day 0 (shown by white circle) was confirmed by imaging at 40× magnification with a Solentim Cell Metric microscope (FIG. 2). After 9 days and 12 days of culture the wells were imaged at 40× magnification with an Olympus Q Colour 3 microscope (FIG. 2). Single B cells were seeded and cultured in media formulations that included different pairwise combinations of three exogenously-added extracellular matrix proteins (Conditions 1, 2 and 3), and all three proteins (Condition 4). Each of Conditions 1, 2, 3, and 4 exhibited marked expansion as observed at both Day 9 and Day 12. In contrast, no expansion could be readily observed at Day 9 among wells containing a cell seeded into base medium only. The extracellular matrix proteins used to carry out this example could be selected from among a collagen, an ECM1, an E-Cadherin, a laminin, an osteopontin, a fibronectin, a vitronectin, or a SPARC, and preferably from a collagen, an ECM1, a laminin, an osteopontin, and a vitronectin.

### Example 4: Single B Cells Markedly Expand in the Presence of Individual, Exogenously-Added Extracellular Matrix Potein

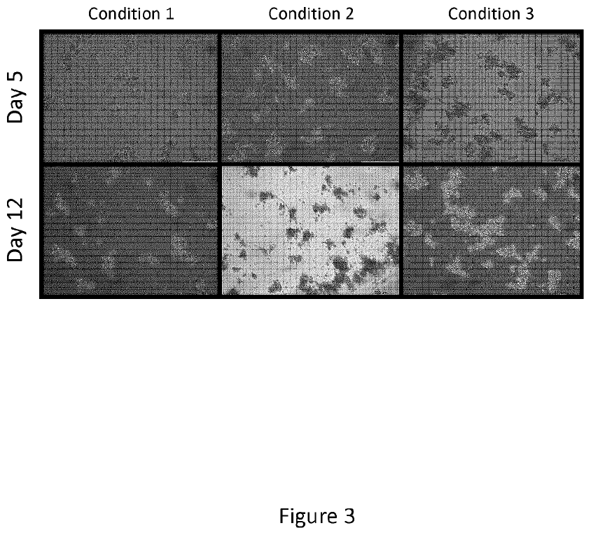

Pan-B cells were enriched and seeded essentially as described in Examples 1 and 2 from a different leukapheresis sample than used in Example 3. After 5 days and 12 days of culture, individual wells were imaged at 40× magnification with an Olympus Q Colour 3 microscope (FIG. 3). Single B cells were seeded and cultured in media formulations that included different, individual, exogenously-added extracellular matrix proteins (Condition 1 and 2), and the combination of both proteins (Condition 3). All tested conditions resulted in expansion of the seeded cells at both time points (FIG. 3). The extracellular matrix proteins used to carry out this example could be selected from among a collagen, an ECM1, an E-Cadherin, a laminin, an osteopontin, a fibronectin, a vitronectin, or a SPARC, and preferably from a collagen, an ECM1, a laminin, an osteopontin, and a vitronectin.

### Example 5: Synergic Effect of Combinations of Extracellular Matrix Protein

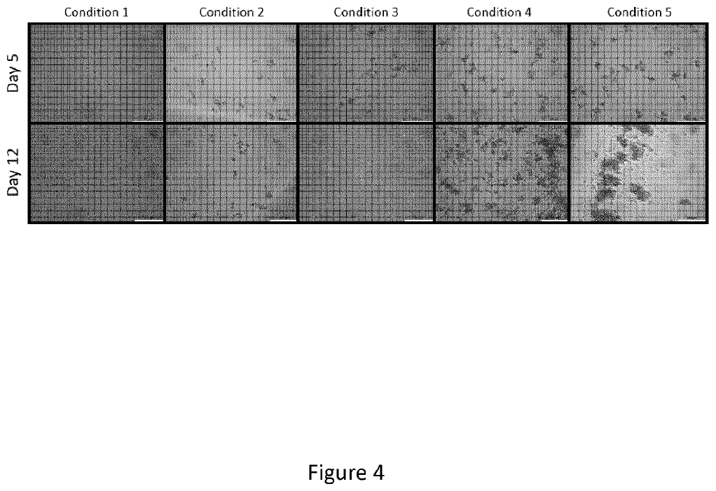

Pan-B cells were enriched and seeded essentially as described in Examples 1 and 2 from a different leukapheresis sample than used in Examples 3 and 4. After 5 days and 12 days of culture the wells were imaged at 40× magnification with an Olympus Q Colour 3 microscope (FIG. 4). Single B cells were seeded and cultured in media formulations that included different, individual, exogenously-added extracellular matrix proteins (Condition 1, 2 and 3), and different pairwise combinations of two proteins (Condition 4 and 5). All tested conditions resulted in expansion of the seeded cells at both time points, though the combination treatments appeared to yield greater expansion (FIG. 4). The extracellular matrix proteins used to carry out this example could be selected from among a collagen, an ECM1, an E-Cadherin, a laminin, an osteopontin, a fibronectin, a vitronectin, or a SPARC, and preferably from a collagen, an ECM1, a laminin, an osteopontin, and a vitronectin.

### Example 6: Enhanced Expansion of Single B Cells in the Presence of a Ligand of Human CD40

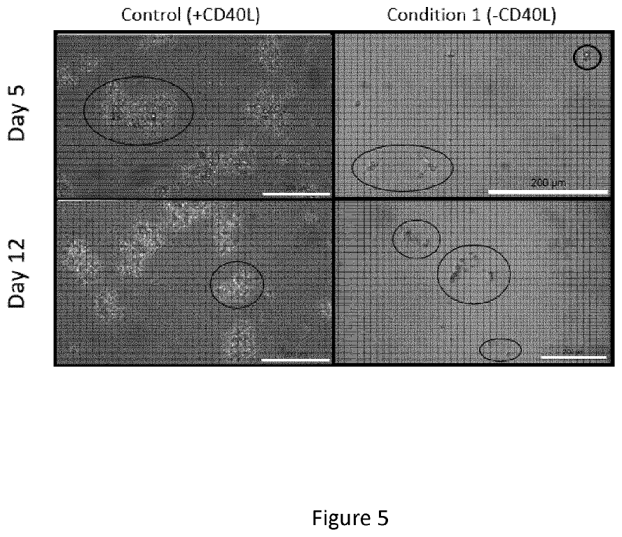

Pan-B cells were enriched and seeded essentially as described in Examples 1 and 2, using cells from the same leukapheresis sample used in Example 5. After 5 days and 12 days of culture the wells were imaged at 40× magnification with an Olympus Q Colour 3 microscope (FIG. 5). Single B cells were seeded into culture medium that included an individual exogenously-added extracellular matrix protein (selected from the preferred list enumerated in Example 2), but either including or lacking an exogenously-added ligand of human CD40. Expansion of seeded single B cells was observed in both conditions and at both time points, but was enhanced in the presence of a ligand of CD40(FIG. 5).

### Example 7: Single B Cells Expand in the Presence of Other Individual, Soluble Extracellular Matrix Protein

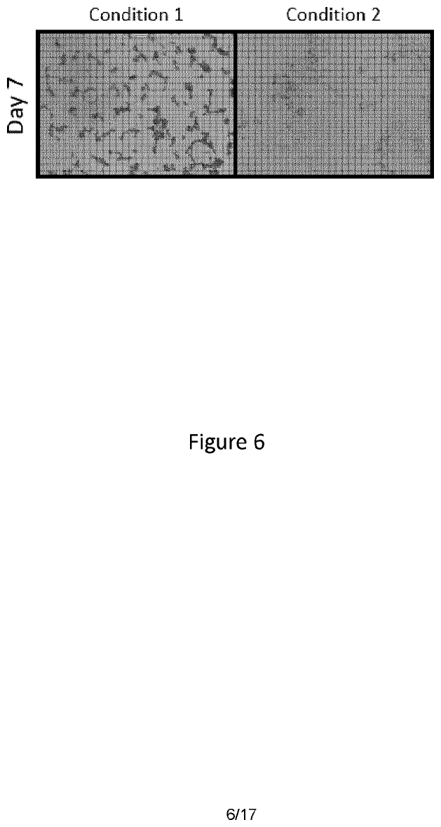

Pan-B cells were enriched and seeded essentially as described in Examples 1 and 2, from the same donor as in Example 3. After 7 days of culture the wells were imaged at 40× magnification with an Olympus Q Colour 3 microscope (FIG. 6). Conditions 1 and 2 included different, individual, exogenously-added extracellular matrix proteins than those tested in previous Examples hereof, but the tested extracellular matrix proteins were nevertheless selected from among: a collagen, an ECM1, an E-Cadherin, a laminin, an osteopontin, a fibronectin, a vitronectin, or a SPARC, and preferably from a collagen, an ECM1, a laminin, an osteopontin, and a vitronectin. . . . Both tested conditions resulted in appreciable expansion of the seeded cells by Day 7 (FIG. 6).

### Example 8: Role of Lipids in the Expansion of Single B Cells

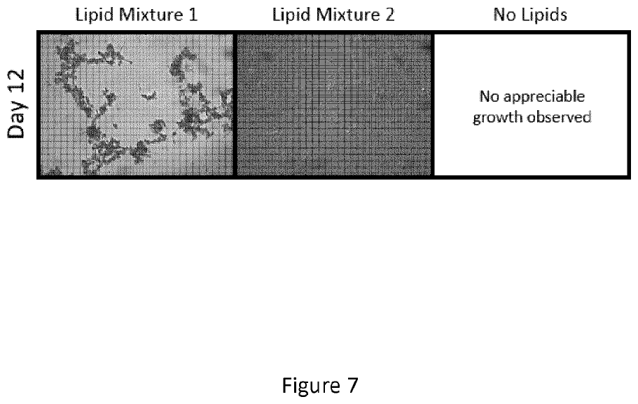

Pan-B cells were enriched and seeded essentially as described in Examples 1 and 2, from the same donor as in Example 3. After 5 and 12 days of culture the wells were imaged at 40× magnification with an Olympus Q Colour 3 microscope (FIG. 7). To assess the role of lipids in B cell expansion, single B cells were seeded into culture media formulations comprising different lipid supplements (animal component free, Lipid Mixture 1 and stabilized, Lipid Mixture 2, both STEMCELL Technologies), but otherwise, including the same single, exogenously-added extracellular matrix protein (selected from the preferred list enumerated in Example 2). After 12 days in culture, single B cells exhibited expansion in the presence of lipids, but no appreciable expansion in the absence of the lipids (FIG. 7).

### Example 9: Differentiation and Activation of Expanded B Cells

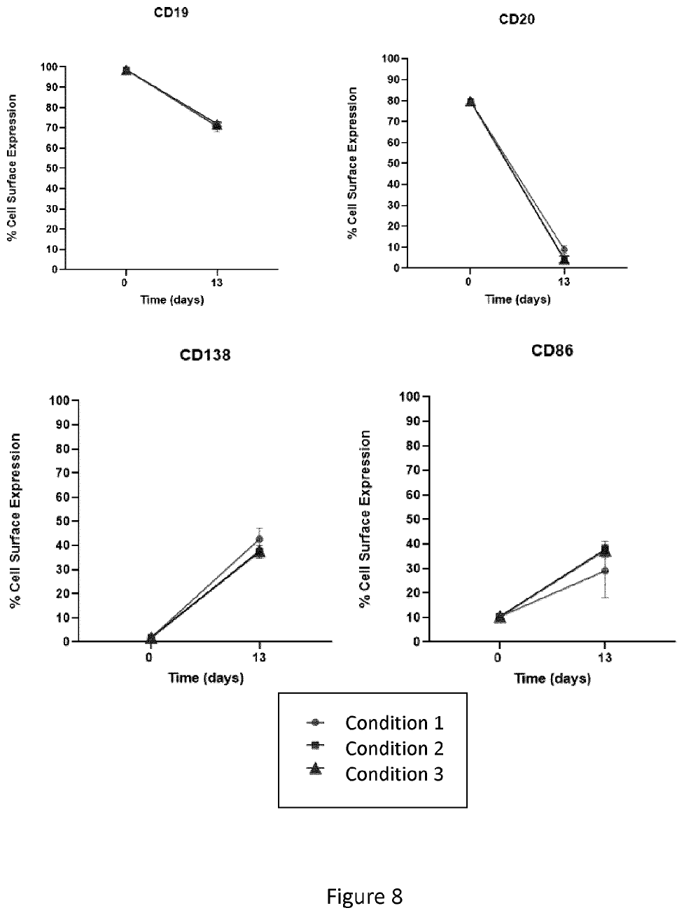

Pan-B cells were enriched and seeded essentially as described in Examples 1 and 2, from the same donor as in Example 3. The cells were expanded in culture for 13 days in culture medium that included different, individual, exogenously-added extracellular matrix proteins (Conditions 1 and 2) (FIG. 8). The extracellular matrix proteins used to carry out this example could be selected from among a collagen, an ECM1, an E-Cadherin, a laminin, an osteopontin, a fibronectin, a vitronectin, or a SPARC, and preferably from a collagen, an ECM1, a laminin, an osteopontin, and a vitronectin.

After the 13-day culture protocol, the arising cells were assessed by flow cytometry for activation and differentiation status using fluorescently labelled antibodies against CD19, CD20, CD86, and CD138 (FIG. 8). In all three conditions tested, seeded pan-B cells activated and differentiated away from primitive phenotypes toward more mature phenotypes. Activation of pan-B cells has been shown by the increased level of B cell activation marker CD86. Differentiation of pan-B cells towards B lineages cells has been shown by the decreased expression levels of CD19 and CD20 surface markers, and increased expression level of the differentiation marker of CD138.

### Example 10: Effect of Cytokines on the Expansion of Single B Cells

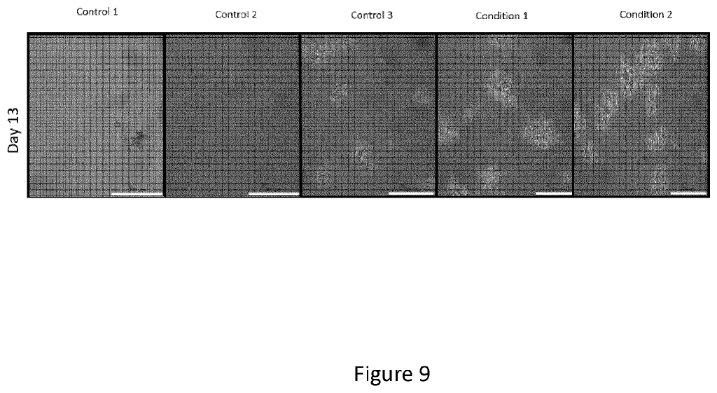

Pan-B cells were enriched and seeded essentially as described in Examples 1 and 2. After 13 days of culture, the wells were imaged at 40× magnification with an Olympus Q Colour 3 microscope (FIG. 9). The extracellular matrix proteins used to carry out this example could be selected from among a collagen, an ECM1, an E-Cadherin, a laminin, an osteopontin, a fibronectin, a vitronectin, or a SPARC, and preferably from a collagen, an ECM1, a laminin, an osteopontin, and a vitronectin.

To assess the role of cytokines in the expansion of single B cells, the cells were seeded into culture media that included an individual, exogenously-added extracellular matrix protein (selected from the preferred list enumerated in Example 2) but differed in terms of the cytokines included therein: IL-2 and IL-10 (Condition 1); and IL-2, IL-4, IL-6, IL-10, and IL-21 (Condition 2). Control conditions included: unsupplemented basal medium (Control 1); basal medium supplemented with cytokines but no exogenously-added extracellular matrix protein (Control 2); and in basal medium supplemented with extracellular matrix protein but no cytokines (Control 3). After 13 days in culture, single B cells exhibited marked expansion in Conditions 1 and 2, but no apparent expansion in control ondition 1 (FIG. 9).

### Example 11: Quantifying the Expansion of Single B Cells

Pan-B cells were enriched and seeded essentially as described in Examples 1 and 2. After 13 days of culture, expanded cells were pelleted to remove supernatant, washed with cold PBS and trypsinized. Cells were then harvested using a cell scraper and were counted using a Nucleocounter to assess expansion from a single B cell. In a first experiment, three different combinations of exogenously-added extracellular matrix proteins were tested and an approximate number of the cells expanded ranged between 2400-15,000 cells/input cell (data not shown). In a second experiment, a single extracellular matrix protein and a combination of two extracellular matrix proteins were tested and an approximate number of the cells expanded ranged between 2000-7000 cells/input cell (data not shown). In a third experiment, a single extracellular matrix protein and a combination of two extracellular matrix proteins were tested and an approximate number of the cells expanded ranged between 15000-46000 cells/input cell (data not shown). Therefore, impressive levels of expansion could be achieved in various media conditions, even accounting for potential difficulties to fully dissociate the expanded cells.

### Example 12: Expansion of Single B Cells in Different Basal Media

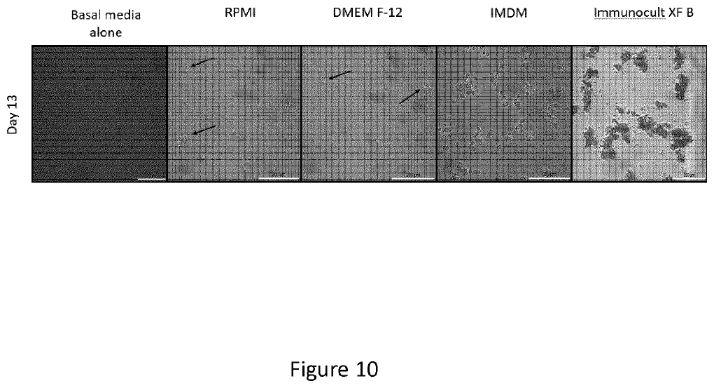

Pan-B cells were enriched and seeded essentially as described in Examples 1 and 2. Single B cells were seeded and cultured in the presence of an individual, exogenously-added extracellular matrix protein (selected from the preferred list enumerated in Example 2) in different commercially available basal media, as indicated in FIG. 10. After 13 days of culture, the cells were imaged at 40× magnification with an Olympus Q Colour 3 microscope (FIG. 10). No growth was observed in the control condition of a basal medium unsupplemented with an extracellular matrix protein. In contrast, appreciable expansion was observed when an exogenously-added extracellular matrix protein was diluted in either RPMI or DMEM F-12 (FIG. 10, white arrows), and marked expansion was observed when diluted in either IMDM or ImmunoCult™ XF (FIG. 10).

### Example 13: Single Human Memory B Cells Expand in the Presence of Exogenously-Added Extracellular Matrix Proteins

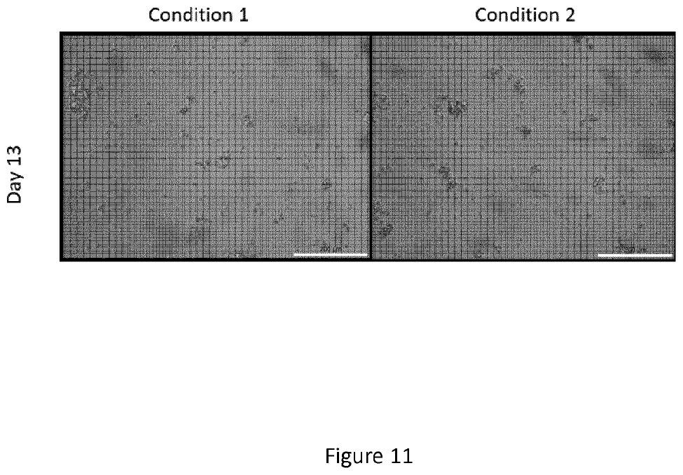

Human memory B cells were enriched and isolated from a leukapheresis sample using the EasySep™ Human Memory B Cell Isolation Kit (STEMCELL Technologies). Single memory B cells were seeded (as described above) into a culture medium that included either two different, exogenously-added extracellular matrix proteins (Condition 1) or three different, exogenously-added extracellular matrix proteins (Condition 2). After 13 days in culture, the cells in a well were imaged at 40× magnification with an Olympus Q Colour 3 microscope (FIG. 11). Appreciable expansion of single memory B cells was observed for both Condition 1 and Condition 2.

### Example 14: Expansion of Single, Human Memory B Cells

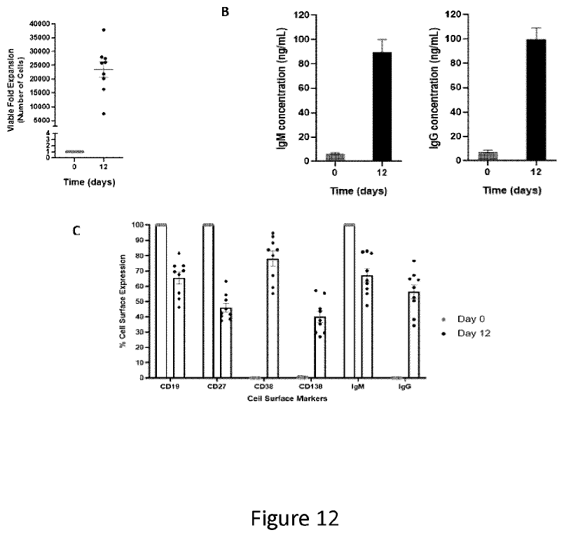

Human pan-B cells isolated from PBMCs as described in Example 1, were stained with CD19, CD27, CD38, CD138, IgM, IgG and DRAQ7 antibodies. On day 0, single viable CD19⁺CD27⁺CD38⁻CD138⁻ IgM⁺ IgG⁻ memory B cells were sorted into respective wells of a 96-well plate (BD FACSAria™ Fusion Cell Sorter), and cultured for up to 4 days in a medium as encompassed by Example 2 comprising an individual, exogenously-added extracellular matrix protein. On day 5, each well of cells was transitioned into a medium formulation essentially as encompassed by Example 2 except that it was not supplemented with an exogenously-added extracellular matrix protein. From day 5 onward, full medium changes occurred every 2-3 days. After 12 total days in culture, the output cells were harvested and counted (essentially as described in Example 11), and viable fold expansion of cells was calculated as 25,000±1549 (FIG. 12A). Day 12 expanded human cells were also tested by ELISA, and appreciable secretion of antibody markers IgM and IgG was detected (FIG. 12B). Last, expression of various cell surface markers by day 0, sorted single cells, and day 12 expanded human cells was assessed by flow cytometry. The results show that single memory B cells expand into a population of B cells comprising memory B cells, antibody secreting cells, and plasmablasts (FIG. 12C). The results also showed that among day 12 expanded cells: i) increased expression of human B cell marker CD38, which correlates with an increase in antibody secreting cells; ii) increase in antibody marker IgG along with a decrease in IgM signifying B cell class switching; iii) increase in activation marker CD86 (not shown); and iv) increase in differentiation marker CD138 showing differentiation of memory B cells to plasmablasts.

### Example 15: Expansion of Single, Human Plasmablasts

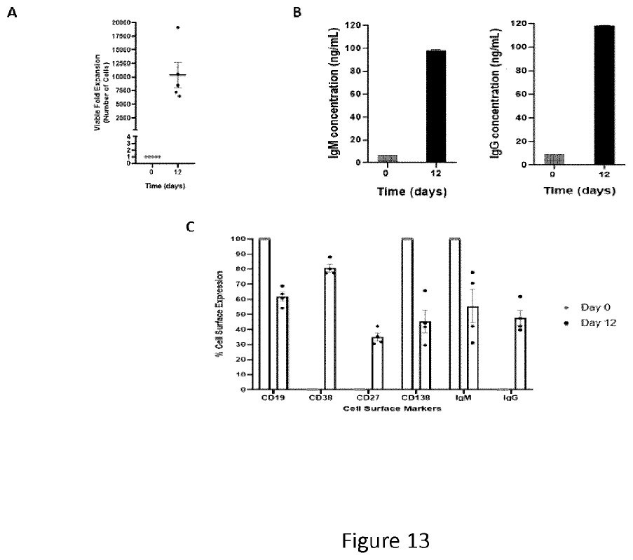

Human pan-B cells were isolated and stained with antibodies essentially as described in Example 14. On day 0, single viable CD19⁺CD27⁻CD138⁺ human plasmablasts were sorted into respective wells of a 96-well plate, and cultured as described in Example 14. After 12 total days in culture, the output cells were harvested and counted (essentially as described in Example 11), and viable fold expansion of cells was calculated as 9,000±1645 (FIG. 13A). Day 12 expanded human cells were also tested by ELISA, and appreciable secretion of antibody markers IgM and IgG was detected (FIG. 13B). Last, expression of various cell surface markers by day 0, sorted single cells, and day 12 expanded human cells was assessed by flow cytometry. The results show that single human plasmablasts expand into a population of B cells comprising memory B cells, antibody secreting cells, and plasmablasts (FIG. 13C). The results also showed that among day 12 expanded cells: i) increased expression of human plasmablast markers CD38 and CD27 signifying an increase in antibody secreting cells; ii) increase in antibody marker IgG along with a decrease in IgM signifying B cell class switching; and iii) decreased expression of differentiation marker CD138.

### Example 16: Single, Mouse Pan-B Cells Expand in the Presence of Exogenously-Added Extracellular Matrix Proteins

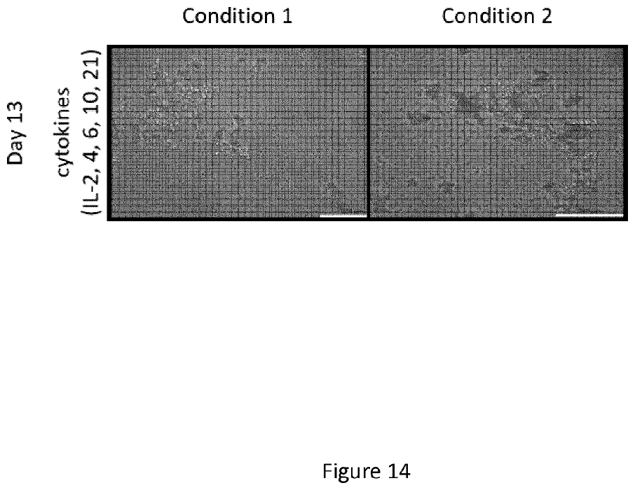

Mouse pan-B cells were enriched from a C57 BL/6 mouse using the EasySep™ Mouse Pan B Isolation Kit (STEMCELL Technologies). On day 0, single B-cells were sorted into respective wells of a 96-well plate (BD FACSAria™ Fusion Cell Sorter), and cultured in a medium formulation, essentially as described in Example 2, comprising one exogenously added extracellular matrix protein (selected from the preferred list enumerated in Example 2). After 13 days in culture, the cells in a well were imaged at 40× magnification with an Olympus Q Colour 3 microscope (FIG. 14). Mouse single B-cells appeared to expand in both conditions tested.

### Example 17: Expansion of Single, Mouse Memory-B Cells

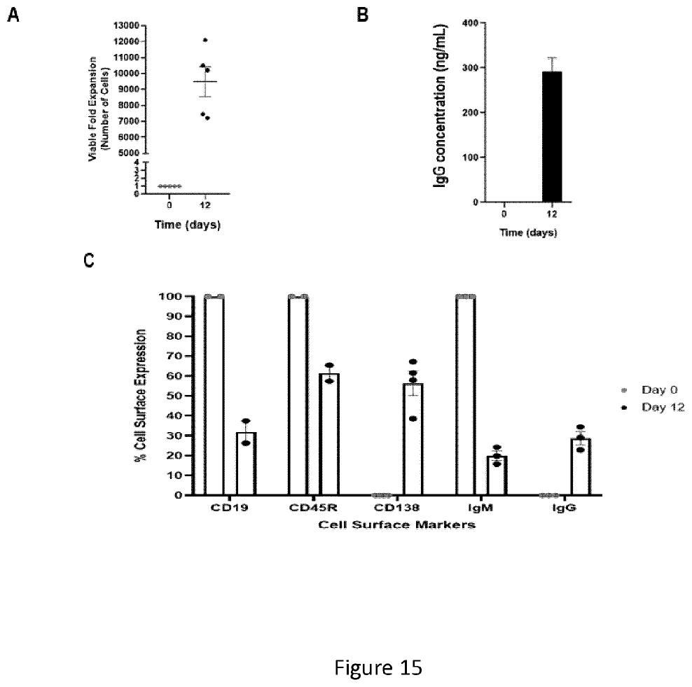

Mouse pan-B cells enriched and isolated from a C57 BL/6 mouse using the EasySep™ Mouse Pan B Isolation Kit (STEMCELL Technologies), were stained with CD19, CD45R, CD138, and DRAQ7 antibodies. On day 0, single viable CD19⁺CD45R^{+CD}138⁻ mouse memory B cells were sorted into respective wells of a 96-well plate (BD FACSAria™ Fusion Cell Sorter), and cultured for up to 4 days in a medium as encompassed by Example 16 comprising an individual, exogenously-added extracellular matrix protein. On day 5, each well of cells was transitioned into a medium formulation essentially as encompassed by Example 2 except that it was not supplemented with an exogenously-added extracellular matrix protein. From day 5 onward, full medium changes occurred every 2-3 days. After 12 total days in culture, the output cells were harvested and counted (essentially as described in Example 11), and viable fold expansion of cells was calculated as 10,000±1423 (FIG. 15A). Day 12 expanded mouse memory B cells were also tested by ELISA, and appreciable secretion of antibody marker IgG was detected (FIG. 15B). Last, expression of various cell surface markers by day 0, sorted single cells, and day 12 expanded mouse cells were assessed by flow cytometry. The results show that single memory B cells expand into a population of B cells comprising memory B cells, antibody secretin cells, and plasmablasts (FIG. 15C). The results also showed that among day 12 expanded cells: i) decrease in CD45R and increase in CD138 suggesting memory cell differentiation to plasmablasts; ii) increase in antibody marker IgG along with a decrease in IgM signifying B cell class switching; and iii) increase in differentiation marker CD138.

### Example 18: Expansion of Single, Mouse Plasmablasts

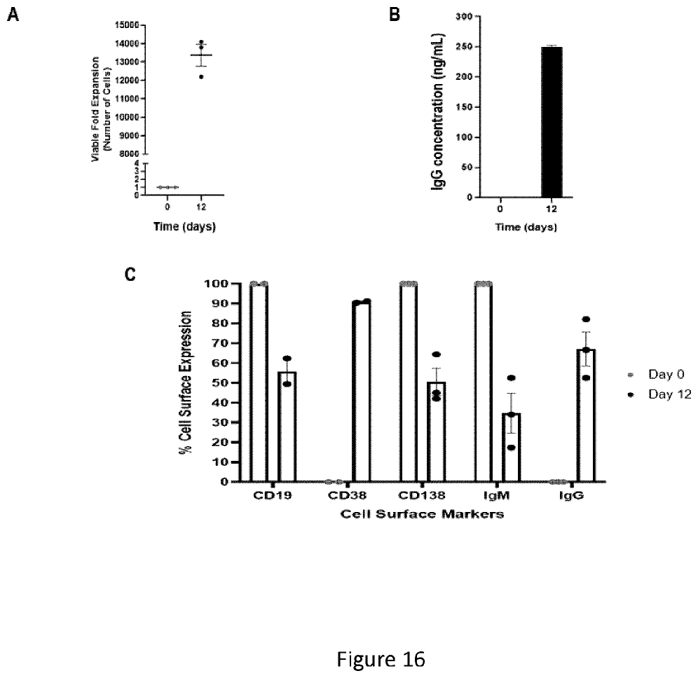

Mouse pan-B cells were isolated and stained with antibodies essentially as described in Example 17. On day 0, single viable CD19⁺CD138⁺ mouse plasmablasts were sorted into respective wells of a 96-well plate (BD FACSAria™ Fusion Cell Sorter), and cultured as described in Example 17. After 12 total days in culture, the output cells were harvested and counted (essentially as described in Example 11), and viable fold expansion of cells was calculated as 13,000±600 (FIG. 16A). Day 12 expanded mouse cells were also tested by ELISA, and appreciable secretion of antibody marker IgG was detected (FIG. 16B). Last, expression of various cell surface markers by day 0, sorted single cells, and day 12 expanded mouse cells was assessed by flow cytometry. The results show that single memory B cells expand into a population of B cells, the population comprising memory B cells, antibody secreting cells, and plasmablasts (FIG. 16C). The results also showed that among day 12 expanded cells: i) increased expression of plasmablast marker CD38 signifying an increase in antibody secreting cells; ii) increase in antibody marker IgG along with a decrease in IgM signifying B cell class switching; and iii) decreased expression of differentiation marker CD138.

### Example 19: Expansion of Single T Cells

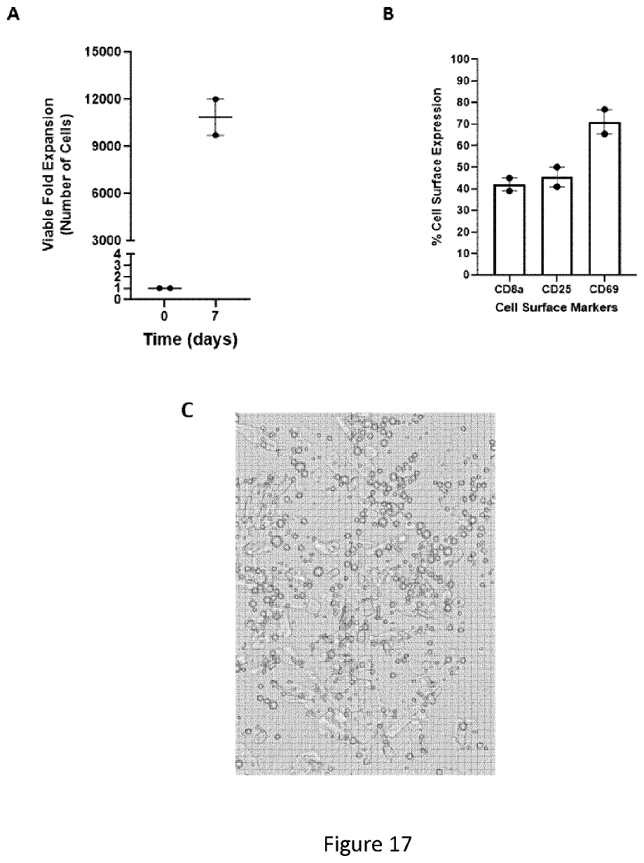

Mouse splenocytes were isolated from a C57 BL/6 mouse and stained with CD3 and DRAQ7 antibodies. On day 0, single viable CD3⁺ mouse T cells were sorted into respective wells of a 96-well plate (BD FACSAria™ Fusion Cell Sorter), and cultured for up to 4 days in a medium as encompassed by Example 16 comprising an individual, exogenously-added extracellular matrix protein. On day 5, each well of cells was transitioned into a medium formulation essentially as encompassed by Example 2 except that it was not supplemented with an exogenously-added extracellular matrix protein. From day 5 onward, full medium changes occurred every 2-3 days. 7 days after transitioning to the second culture environment (12 total days), the output cells were harvested and counted (essentially as described in Example 11), and viable fold expansion of cells was calculated as >10,000±1423 (FIG. 17A). Also, after 12 total days in culture the output cells were analyzed by flow cytometry for surface expression of CD8a (a T cell marker), CD25 (a T cell activation marker), and CD69 (a T cell activation marker) (FIG. 17B). A similar workflow as described in this example was performed on sorted human T cells after staining a sample of PBMC with CD3 and DRAQ7 antibodies. FIG. 17C depicts the scale of expansion of a single, viable human CD3⁺ T cell sorted into a well of 96-well plate, and cultured for 12 days in a medium essentially as described in Example 2.

1. A cell culture media supplement for expanding mammalian lymphocytes or progenitors thereof, the supplement comprising:
one or more of or two or more of: a ligand of CD40, a mixture of lipids, and one or more cytokines; and
a diluent.

2-3. (canceled)

4. The supplement of claim 1, further comprising a first type of an extracellular matrix protein, a first type of an extracellular matrix protein and a second type of extracellular matrix protein, or a first type of an extracellular matrix protein, a second type of extracellular matrix protein, and a third type of extracellular matrix protein.

5.-6. (canceled)

7. The supplement of claim 4, wherein the extracellular matrix protein is selected from the group consisting of a collagen, an ECM1, an E-Cadherin, a laminin, an osteopontin, a fibronectin, a vitronectin, or a SPARC.

8.-10. (canceled)

11. The supplement of claim 1, wherein the ligand of CD40 comprises one or more proteins.

12. The supplement of claim 1, wherein the one or more cytokines are selected from IL-2, IL-4, IL-6, IL-10, and IL-21.

13. (canceled)

14. The supplement of claim 1, wherein the supplement is serum-free.

15.-18. (canceled)

19. A culture medium for expanding mammalian lymphocytes or progenitors thereof, the medium comprising:
a basal medium; and
one or more of or two or more of: a ligand of CD40, a mixture of lipids, and one or more cytokines.

20. (canceled)

21. The culture medium of claim 19, further comprising a first type of an extracellular matrix protein.

22.-23. (canceled)

24. The culture medium of claim 21, wherein the extracellular matrix protein is selected from the group consisting of a collagen, an ECM1, an E-Cadherin, a laminin, an osteopontin, a fibronectin, a vitronectin, or a SPARC.

25.-26. (canceled)

27. The culture medium of claim 19, wherein the one or more cytokines are selected from IL-2, IL-4, IL-6, IL-10, and IL-21.

28. The culture medium of claim 19, wherein the ligand of CD40 comprises one or more proteins.

29.-30. (canceled)

31. The culture medium of claim 19, wherein the culture medium:
a) is serum-free and/or
b) supports feeder-free expansion of lymphocytes and progenitors thereof; and/or
c) supports animal component-free expansion of lymphocytes and progenitors thereof.

32.-33. (canceled)

34. A method of expanding mammalian lymphocytes or progenitors thereof, the method comprising:
seeding the lymphocytes or progenitors thereof into a culture environment comprising one or more of a ligand of CD40, a mixture of lipids, and one or more cytokines; and
incubating the seeded lymphocytes or progenitors thereof in the culture environment for more than one day.

35. The method of claim 34, further comprising a first type of an extracellular matrix protein in the culture environment, a first type of an extracellular matrix protein and a second type of extracellular matrix protein in the culture environment, or a first type of an extracellular matrix protein, a second type of extracellular matrix protein, and a third type of extracellular matrix protein in the culture environment.

36.-37. (canceled)

38. The method of claims 35, wherein the extracellular matrix protein is one or more of a collagen, an ECM1, an E-Cadherin, a laminin, an osteopontin, a fibronectin, a vitronectin, or a SPARC.

39.-41. (canceled)

42. The method of claim 34, wherein the ligand of CD40 comprises one or more proteins.

43. The method of claim 34, wherein the one or more cytokines are selected from IL2, IL4, IL6, IL10, and IL21.

44. The method of claim 34, wherein the mixture of lipids is chemically defined.

45. The method of claim 34, wherein seeding and incubating are in feeder-free conditions.

46.-48. (canceled)

49. The method of claim 34, further comprising differentiating and/or activating the lymphocytes or progenitors thereof in the culture environment.

50. (canceled)

51. The method of claim 34, wherein the lymphocytes or progenitors thereof are seeded as single cells or at a clonal density.
